(19) United States
(12) Patent Application Publication
(10) Pub. No.: US 2025/0049606 A1
(43) Pub. Date: Feb. 13, 2025

(54) INSERT WITH VISCOUS FILLER FOR ENDOSCOPY SUPPORT

(71) Applicant: ColoWrap, LLC, Durham, NC (US)

(72) Inventors: James HATHORN, Carrboro, NC (US); Wayne HENDERSON, Carrboro, NC (US); Anjana Mohanty, Durham, NC (US); Catherine Froelicher, Raleigh, NC (US)

(21) Appl. No.: 18/799,999

(22) Filed: Aug. 9, 2024

Related U.S. Application Data
(60) Provisional application No. 63/519,179, filed on Aug. 11, 2023.

(51) Int. Cl. A61F 5/03; A61B 1/31; A61F 5/32; A61F 5/34; A61F 5/37

(52) U.S. Cl. CPC A61F 5/03; A61F 5/32; A61F 5/34; A61F 5/37; A61B 1/31

(57) ABSTRACT

A method and apparatus for placement at an abdomen of a patient in connection with an endoscopy procedure, comprising: an outer housing having at least a portion comprising a flexible, pliable material; and an inner material within the outer housing, wherein the inner material has at least one property related to adipose tissue.

### CROSS REFERENCE TO RELATED APPLICATION(S)

This application claims the benefit of and priority to U.S. Provisional Application Ser. No. 63/519,179, entitled “Insert with Viscous Filler for Endoscopy Support” and filed on Aug. 11, 2023, which is expressly incorporated by reference herein in its entirety.

### INTRODUCTION

A colonoscopy is an example of an endoscopy procedure including an examination of the large intestine or colon through the use of a colonoscope. A colonoscope is a flexible, tube-like inspection device having a camera at its end. Colonoscopies are performed for a variety of medical reasons including detection of inflamed tissue, ulcers, abnormal growths or polyps, and colorectal cancer. Colonoscopy is increasingly used as a screening tool to detect colorectal cancer.

During a colonoscopy, as an example of an endoscopy procedure, a colonoscope is inserted into a patient's rectum and then advanced to the beginning of the colon (an area known as the cecum) in order to examine the lining of the large intestine. The efficiency and accuracy of this procedure is largely dependent on the ease with which the colonoscope can be advanced. During the procedure, the colon may become over-distended or flopped in unnatural directions creating loops that hinder the advancement of the colonoscope and resulting in patient discomfort, longer examination times, and potentially inaccurate or incomplete screenings.

Currently, the difficulty in advancing the scope is addressed by the application of manual pressure by a technician to manually support the patient's colon. The application of manual pressure is time-consuming and varies depending on the particular technician's strength, technique, endurance, and training. In order to apply differential pressure or to change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure places a physical toll on the technician.

### SUMMARY

In an aspect of the disclosure, a method and apparatus for applying pressure to the abdomen of a patient is provided to case the passage of an endoscope during procedures used to examine the bowels including colonoscopy, sigmoidoscopy, and enteroscopy. Aspects presented herein exert both broad, uniform lower abdominal pressure as well as additional, location-specific pressure upon specific parts of the colon including the sigmoid and transverse, and/or small bowel to assist with the advancement, withdrawal, and/or visualization as part of an endoscopy, colonoscopy, sigmoidoscopy, or enteroscopy procedure. Aspects presented herein may assist in preventing and reducing intestinal looping, eliminating the need for the application of manual pressure, improving patient safety, comfort, and satisfaction, and preventing musculoskeletal injury to endoscopy healthcare providers. Aspects may improve visualization and/or withdrawal portions of the procedure.

Aspects include an insert that can be used in connection with an support device that applied pressure through compression. The insert may help in the application of the compression from the device to the abdomen of the patient and may assist in applying a more consistent amount of support to patients of varying body mass indexes. In some aspects, the insert may assist in maintaining a position of the sigmoid and/or transverse regions of the colon during an endoscopy procedure. In some aspects, the insert may be configured with a density similar to adipose tissue to help communicate the compression applied by a support device to the colon of the patient to assist in an endoscopy procedure.

In some aspects, an apparatus for placement at an abdomen of a patient in connection with an endoscopy procedure, includes an outer housing having at least a portion comprising a flexible, pliable material; and an inner material within the outer housing, wherein the inner material has at least one property related to adipose tissue.

In some aspects, a method of applying an insert at a patient in connection with an endoscopy procedure, the includes: placing the insert between the patient and at least a portion of an elastic compression device, wherein the insert includes: an outer housing having at least a portion comprising a flexible, pliable material; and an inner material within the outer housing, wherein the inner material has at least one property related to adipose tissue; maintaining or applying at least the portion of the elastic compression device over the insert; and maintaining the insert at an abdomen of the patient during one or more of endoscope insertion, imaging, endoscope withdrawal, or a post endoscopy time period.

Additional advantages and novel features of aspects of the present invention will be set forth in part in the description that follows, and in part will become more apparent to those skilled in the art upon examination of the following or upon learning by practice thereof.

### BRIEF DESCRIPTION OF THE DRAWINGS

FIG. 1A is a schematic view of a colon with an endoscope (also known as a colonoscope for colonoscopy procedures) partially inserted therein.

FIG. 1B is a schematic view of a colon in which a sigmoid loop has developed due to an attempt to advance the endoscope against an unsupported colon wall;

FIG. 1C is a schematic view of a colon showing the application of manual pressure to the colon to facilitate insertion of an endoscope.

FIG. 2 is an illustration of an unfastened endoscopy band device with an elastic secondary strap capable of retaining tension and exerting directed force to the colon, including the sigmoid, traverse, and cecal regions, stretched and fastened to the primary wrap.

FIG. 3A is a perspective view of the endoscopy band device depicted in FIG. 2 as it appears when applied to a patient, in accordance with aspects of the present invention.

FIGS. 3B and 3C illustrate examples of a technician applying manual pressure to the abdomen of a patient.

FIG. 4 illustrates a method of use of an endoscopy band device with an elastic secondary strap capable of retaining tension and exerting directed force for an endoscopy procedure.

FIG. 5 is an illustration of a portion of an endoscopy band having multiple secondary straps.

FIG. 6 is an endoscopy band with multiple secondary straps applied to a patient.

FIG. 7 illustrates a pelvic region of a patient, including an iliac crest.

FIG. 8A and FIG. 8B illustrate an example of a filled pad or insert that can be applied with a compression wrap.

FIG. 9 illustrates various views of an example filled pad or insert that can be applied with a compression wrap.

FIG. 10 illustrates a view of an example filled pad or insert from a side that is positioned opposite a patient.

FIG. 11 illustrates a view of an example filled pad or insert from a side that is positioned toward a patient.

FIG. 12 illustrates a side view of an example filled pad or insert that can be applied with a compression wrap.

FIG. 13, FIG. 14, and FIG. 15 illustrate examples of an example filled pad or insert having added particles, such as weighted beads, added with a viscous filler.

FIG. 16 illustrates an example of a filled pad or insert having multiple, separate chambers.

FIG. 17 illustrates an example of a filled pad or insert having multiple, connected chambers.

FIG. 18 illustrates an example of a filled pad or insert including a component to provide heating.

FIG. 19 illustrates an example of a filled pad or insert having different sizes.

FIGS. 20-23 illustrate examples of a filled pad or insert placed on the primary wrap and under the secondary straps of the example endoscopy support device.

FIG. 24A and FIG. 24B illustrate example aspects of methods of use of a filled pad or insert with an endoscopy support apparatus during an endoscopy procedure.

### DETAILED DESCRIPTION

Aspects include an insert that can be used in connection with an support device that applied pressure through compression. The insert may help in the application of the compression from the device to the abdomen of the patient and may assist in applying a more consistent amount of support to patients of varying body mass indexes. In some aspects, the insert may assist in maintaining a position of the sigmoid and/or transverse regions of the colon during an endoscopy procedure. In some aspects, the insert may include a density similar to adipose tissue to help communicate compression provided by a band around the lower abdomen to the colon in order to prevent looping and assist in insertion, withdrawal and/or visualization during an endoscopy procedure.

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

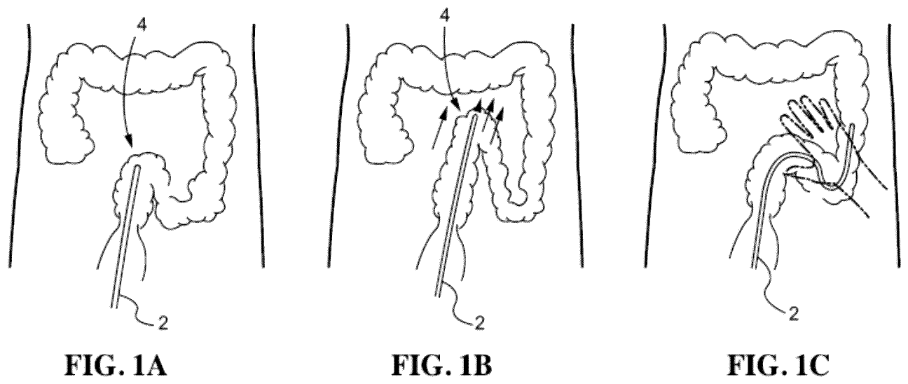

FIGS. 1A-1C, illustrate a sequence of steps of a colonoscopy, as one example of an endoscopy procedure. In FIG. 1A, a colonoscope 2 is inserted into the patient's rectum and advanced forward through the length of the colon. As the operator passes the colonoscope through the sigmoid region of the colon 4, the colonoscope may become impinged and cause distention and looping of the anatomy, as shown in FIG. 1B. The distention causes discomfort to the patient and increases the time required for the colonoscopy. In order to reduce the distended or looped area, a technician may apply manual pressure to abdomen of the patient. Among other examples, the technician may be a nurse, assistant, or other staff member. For example, the pressure may be applied by a nurse or surgical assistant as shown in FIG. 1C.

The application of manual pressure is time-consuming and places a physical toll on the technician. The effectiveness of the manual pressure varies depending on the particular technician's strength, technique, endurance, and training. In order to apply differential pressure and change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure may lead to injury of the patient or of the technician.

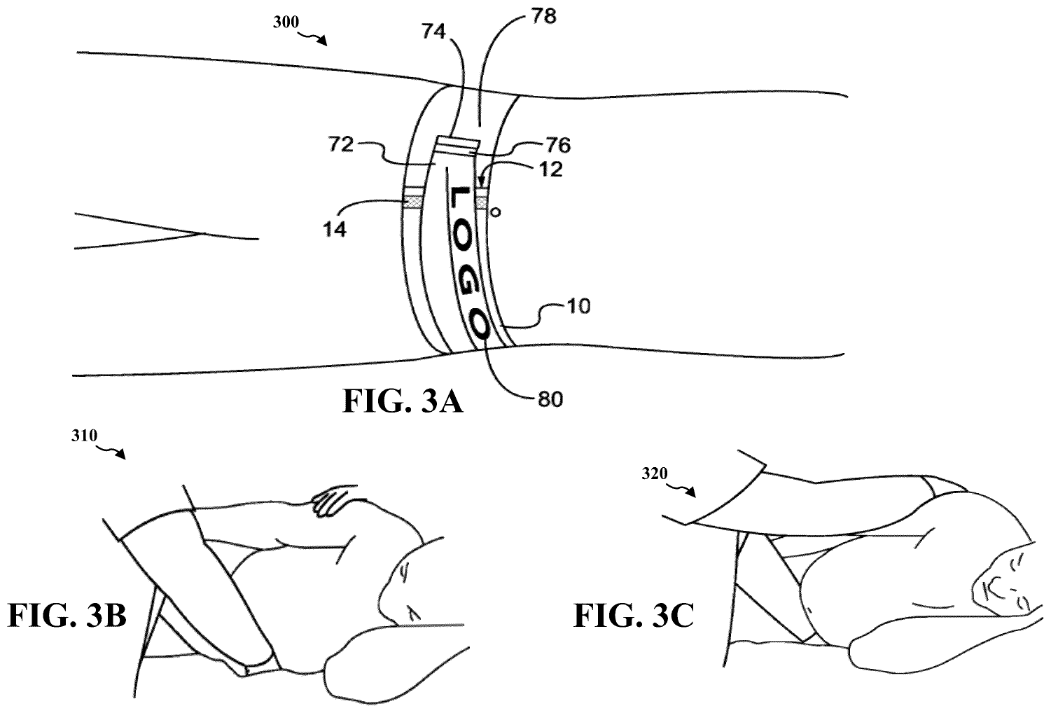

Many patients undergo colonoscopy while placed in the left lateral decubitus position on the stretcher or operating table. FIG. 3B and FIG. 3C illustrate examples 310 and 320 of a technician applying manual abdominal pressure when the patient is placed in a left lateral decubitus position. Additional information about the use of such manual pressure can be found in Prechel J A, Hucke R. Safe and effective abdominal pressure during colonoscopy: forearm versus open hand technique. Gastroenterol Nurs 2009; 32:27-30; quiz 31-2, the entire contents of which are incorporated herein by reference. In applying manual pressure, the technician may reach over the patient from the opposite side of the table and to deploy pressure by placing their hands against the patient's sigmoid colon and then leaning backwards, using their bodyweight for leverage to exert force. While these methods are generally effective at generating pressure, they have also been identified as a causative factor for the high rate of work-related injuries among endoscopy nurses and staff. Physicians performing colonoscopy suffer work-related musculoskeletal injury at a particularly high-rate as well. The most frequent site of physician injury is the right upper extremity which experiences peak torque forces when while operators are attempting to advance the scope through (a looping) sigmoid colon. Additional details can be found in Spanarkel M, Hathorn J P. Looping During Colonoscopy: A Major, Implied Cause of Injury Among Endoscopy Healthcare Providers and a Proposed Solution, 2013, the entire contents of which are incorporated herein by reference.

Aspects described herein may similarly be applied for other endoscopic procedures such as, but not limited to, sigmoidoscopy and retrograde enteroscopy procedures. Sigmoidoscopy is an examination of only the lower part of the colon, from the anus to the descending colon. An endoscope is inserted into the lower part of the colon. Enteroscopy is an examination of the small bowel. During retrograde enteroscopy, an endoscope is inserted in the anus and passed through the colon and the cecum and into the small bowel. Successfully navigating the loop-prone sigmoid region is necessary to complete both sigmoidoscopy and retrograde enteroscopy and thus aspects described herein can be used to help facilitate colonoscopy, sigmoidoscopy, retrograde enteroscopy, and other endoscopic procedures. The small bowel is part of the intestine between the stomach and the colon. The small bowel comprises three segments, the duodenum, the jejunum, and the ileum. It may be difficult to advance an endoscope in order to visualize the patient's entire small bowel due to the length, tortuosity/redundancy of the small bowel.

The teachings of each of U.S. application Ser. No. 17/180,676, entitled Endoscopy Band with Visual Indicator to Assist Placement, and filed on Feb. 19, 2021; U.S. application Ser. No. 16/818,877, entitled Endoscopy Band with Sigmoid Support Apparatus, and filed on Mar. 13, 2020; U.S. application Ser. No. 15/256,019, entitled “METHOD AND APPARATUS FOR ENHANCED VISUALIZATION DURING ENDOSCOPY,” and filed on Sep. 2, 2016; U.S. Provisional Application No. 62/214,747, entitled “IMPROVED BOWEL STABILITY AND ENHANCED VISUALIZATION DURING ENDOSCOPY” and filed on Sep. 4, 2015; and U.S. application Ser. No. 14/575,860, entitled “ENDOSCOPY BAND WITH SIGMOID SUPPORT APPARATUS,” and filed on Dec. 18, 2014; U.S. application Ser. No. 13/344,715, entitled “METHOD AND APPARATUS FOR TENSILE COLONOSCOPY COMPRESSION,” and filed on Jan. 6, 2012; U.S. Provisional Application Ser. No. 61/917,469, entitled “COLONOSCOPY BAND WITH SIGMOID SPLINT” and filed on Dec. 18, 2013; U.S. Provisional Application Ser. No. 61/944,658 entitled “ENDOSCOPY BAND WITH SIGMOID SUPPORT APPARATUS” and filed on Feb. 26, 2014; U.S. Provisional Application Ser. No. 62/978,797, entitled “Endoscopy Band With Visual Indicator to Assist Placement” and filed on Feb. 19, 2020, are expressly incorporated by reference herein in their entirety.

In order to prevent looping and assist in insertion and/or withdrawal of an endoscope, and/or to improve imaging during an endoscopy procedure, a compression device may be placed on the patient. The device may include a primary elongated band or wrap of sufficient length for placement around a patient's lower abdomen. The primary wrap may include a closing mechanism, such as a hook and loop fastener material to secure the device around the patient and to apply an amount of broad support and compression. The device may also include one or more secondary straps that enable the technician to adjust the amount of compression applied by the device.

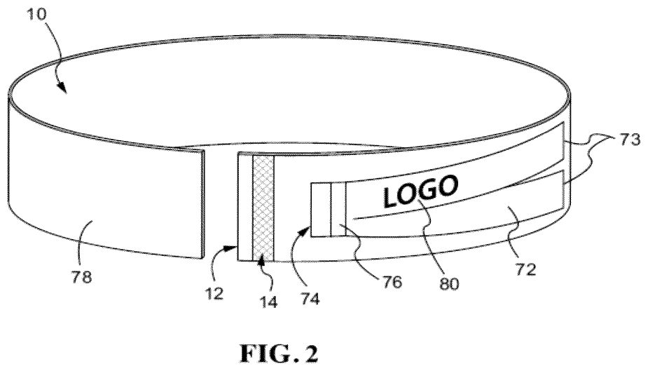

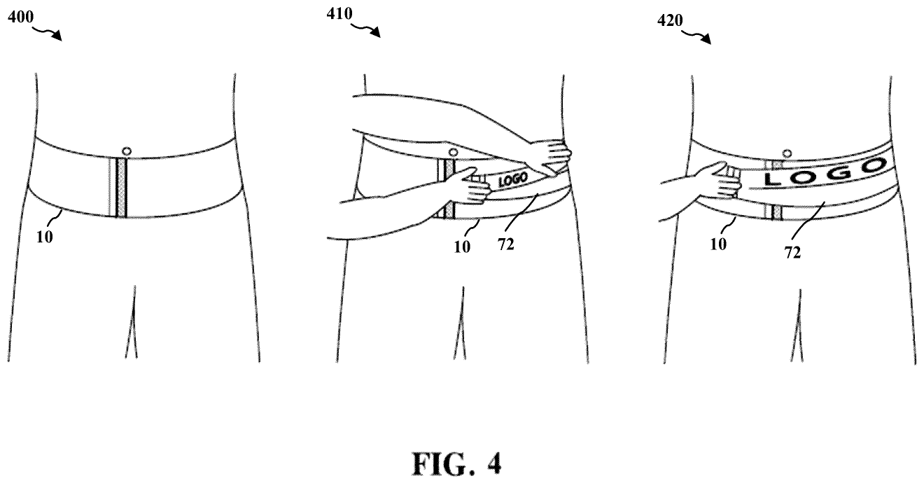

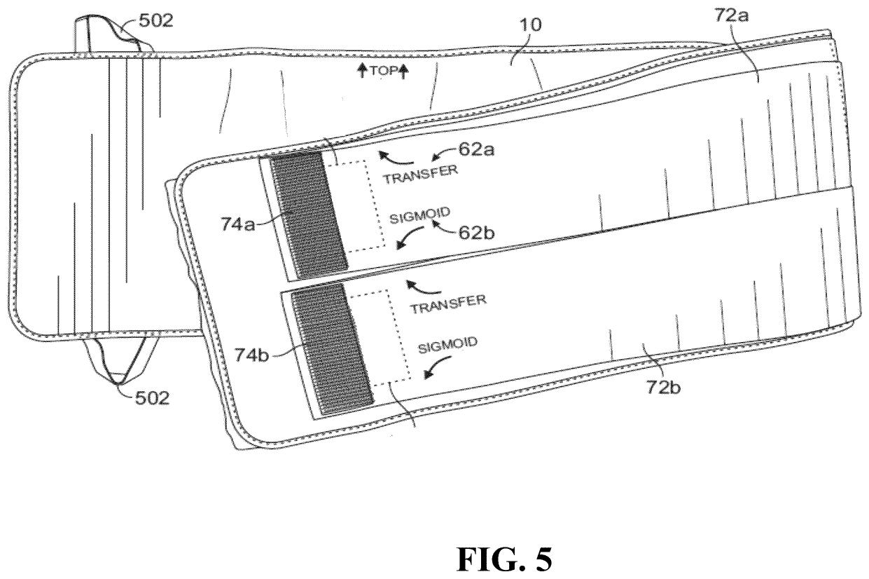

FIG. 2 illustrates an example device including a primary elongated band or wrap 10 of sufficient length for placement around a patient's lower abdomen. A closing mechanism 12 may be provided at the end of the primary band to secure the device around the patient so that it provides the desired amount of broad support and compression. A handle 14 may be sewn onto the exterior of one or both ends of the primary wrap to assist in fastening and closure. FIG. 4 illustrates an example application 400 showing a primary elongated band or wrap 10 wrapped around an abdomen of a patient. FIG. 4 shows at 410 and 420, a secondary strap 72 being pulled, or stretched, to apply added compression to at least a portion of the abdomen. FIG. 5 illustrates a portion of an example wrap 10 having handles 502 provided at an end of the wrap 10. The handles may be used by the technician to stretch the wrap and apply a desired degree of compression when fastening the primary wrap 10 around the patient. In some aspects, an extension component, or offset piece, may extend from the primary wrap 10, and the handles 502 may be formed as a loop of material that is placed at the respective extensions. In some aspects, an angled placement of the handles or finger loops may improve the leverage of the user in pulling the edge of the primary wrap underneath the opposite edge in order to apply compression before fastening the opposite edge to the primary wrap, e.g., using a hook and loop or hook and pile type fastener, in order to maintain the compression on the abdominal region of the patient.

The primary wrap 10 may have a circumferential length between approximately 15 and 75 inches in order to accommodate varying abdominal girths in patients. The width of the primary wrap 10 may be between 6 and 10 inches, although variations having widths of between 3 and 20 inches may be used depending on the size of the patient and to accommodate special circumstances such as an abdominal hernia or a large pannus. For example, the primary wrap may be configured to have a width that allows it to be fastened around the patient's lower abdomen with the upper edge of the wrap just below the umbilicus, or near the umbilicus, and the bottom edge of the wrap along or close to the pubic line. The width of the primary wrap may be selected so as not to be so large that the upper edge conceals the umbilicus and additional areas of the abdomen above the umbilicus. This configuration may be made in order to avoid pressure on the diaphragm or stomach, as pressure on the diaphragm and stomach during colonoscopy can increase the risk of oxygen deprivation and aspiration events, respectively, due to the fact that the patient is generally sedated during the procedure. The primary wrap 10 may comprise, entirely or in part, a flexible, bio-compatible foam, rubber, neoprene, polyester, nylon, non-woven or woven fabric, mesh fabric, synthetic fabric, microfiber fabric, silicon or vinyl plastic, or any other materials generally known to be used in medical fabrics and goods. The primary wrap 10 may be composed of both clastic and inelastic materials. In one example, the primary wrap 10 may comprise multiple layers laminated together. For example, the primary wrap 10 may comprise a neoprene layer and an outer fabric layer laminated on the neoprene layer. The fabric layer may enable the secondary strap to be removably fastened along the length of the primary wrap and may also enable visual indicators to be printed on the fabric, such as a nylon loop fabric. The primary wrap may also comprise an inner layer laminated on the neoprene. The inner layer may comprise a fabric or other material that is selected for skin sensitivity. For example, the inner layer may comprise a fabric or other material that is latex free, biocompatible, and/or skin sensitive. The inner layer may be the layer that is applied against the skin of the patient. In some aspects, the primary wrap may comprise 3D spacer fabrics, which may provide compression, elasticity, cushioning, breathability, air permeability, thermal insulation, temperature regulation, flexibility with resistance to bending/rolling, good draping, adjustable vapor transport, good aging qualities, surface and wash resistance, sterilization capabilities, and diverse surface design capabilities. A 3D spacer fabric may comprise a manufactured textile structure in which two fabric layers are connected by a layer of pile threads, which may be referred to as pile yarns.

The primary wrap may comprise two or more sections that vary in material type. In one example, a first set of one or more sections may be composed of a flexible, elastic or semi-elastic, medium-thickness, latex-free neoprene with thin polyester or nylon glued to its interior and exterior sides. Among others, this material may be capable of providing broad, firm, yet comfortable support to the patient's abdominal region. Another set of one or more sections of the primary wrap 10 may comprise a relatively inelastic material, such as a woven fabric. The inelastic section may be provided at the location into which or under which an insert is placed. Upon deployment of the device, the inelastic section may be positioned in the patient's lower left abdominal quadrant, over the sigmoid region.

For the primary wrap to provide appropriate general compression and support, it may be important that the wrap remain flat against the body when fastened around the abdomen. This is notable because certain materials and designs may tend to roll-up when stretched or wrapped around the abdomen, particularly when the device is being applied to patients with a large pannus. To prevent roll-up from occurring, aspects of the invention may include reinforcements to ensure that the primary abdominal wrap remains flat against the body when used in patients of varying body sizes. This may be accomplished by the application of serge stitching along the edges of the primary abdominal band.

The primary wrap may also accommodate an insert or attachment that provides specific support to one or more areas of the colon including the sigmoid, transverse, and cecal regions as well as the splenic and hepatic flexures. When the device is securely fastened, the secondary strap, the insert, or both, may be pushed, pulled, or otherwise pressed into the body in a manner that serves to support or ‘splint’ one or more areas of the colon including the sigmoid, transverse, and cecal regions as well as the splenic and hepatic flexures. Aspects described herein may be designed to provide broad lower abdominal support, and additional direct force to one or more areas of the colon including the sigmoid, transverse, and cecal regions as well as the splenic and hepatic flexures of a patient undergoing colonoscopy.

The primary wrap 10 may be placed around the patient's lower abdomen and secured using a closing mechanism 12 consisting of a strip of VELCRO® or hook material 13 placed on the interior of the wrap 10 close to the location of the handle 14 on the opposite side. This hook strip 13 may be fastened to the exterior side of the opposite end of the primary wrap 10.

The addition of one or more appendages on the exterior of the primary wrap facilitates the application of additional directed force, e.g., without requiring adjustment of the primary wrap. The appendages may comprise one, or multiple, straps attached on one side to the edge or edges of non-elastic section. FIGS. 2, 3A, and 4 illustrate an example with a single secondary strap.

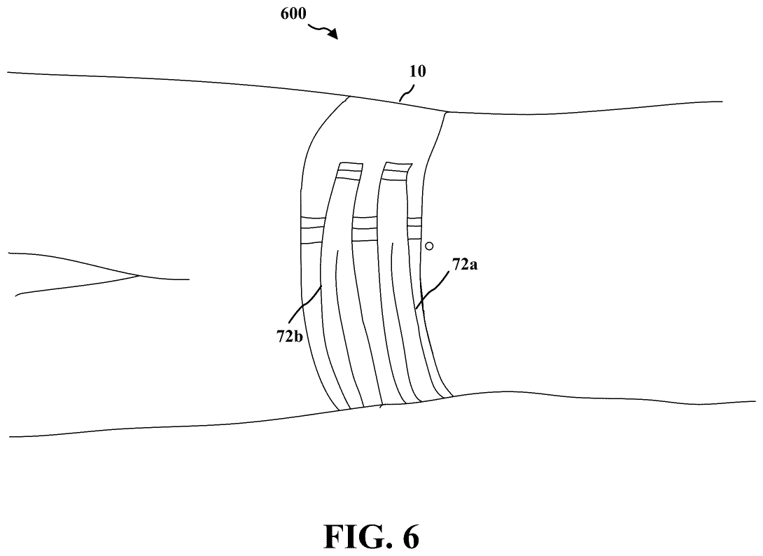

FIG. 5 illustrates an example having multiple secondary straps 72a, 72b. FIG. 6 illustrates an example 600 of a device having multiple secondary straps that is placed on a patient. On the unattached end of these straps, there may be a handle, e.g., 74a, 74b shown in FIG. 5. The handle or loop near the end of the strap may provide greater case to a technician, or added security, in pulling the clastic strap to apply targeted, additional compression. The straps 72a, 72b may be pulled horizontally along the exterior of the primary wrap, and fastened using the closing mechanism securely enough to maintain tension. The one or more straps may comprise an elastic material that is configured to be stretched and fastened to the primary wrap in order to apply targeted compression through elastic contraction of the material. The amount of targeted compression may be adjusted through adjustment of the position at which the removable end is coupled to the primary wrap, such as through a hook and loop type fastener. VELCRO® is an example of a hook and loop type fastener. In some examples, the straps may be secured to an inelastic section of the primary wrap, and the tension generated by fastening these straps may cause additional compression of the inelastic section and/or an insert toward the body of the patient. In some aspects, the device may include visual labels 62a, 62b, e.g., including text and/or arrows that assist with placement and/or application of targeted compression.

As depicted at 300 in FIG. 3A, the secondary strap 72 may allow nurses and technicians to easily adjust and readjust the force on a particular region of the patient's abdomen, such as the sigmoid colon and/or the transverse colon among other example regions, from the location in the procedure or operating room that these staff members typically occupy, relative to how patients are often positioned during an endoscopy procedure. The endoscopy compression device described herein additionally eliminates the need for the nurse or assistant to provide manual abdominal compression, thereby reducing their risk of musculoskeletal injury. Additional, manual compression may be applied along with compression from the device. The device is designed to be quickly and easily removed should the need arise.

The secondary strap 72 may be coupled, e.g., sewn or permanently fastened, to a portion of the wrap that is configured for placement over a left side of the patient's lower abdomen. The portion of the secondary strap 72 that is coupled to the primary wrap may be referred to as the secured end and may be non-removably coupled to the strap, such as sewn or formed as an extension of the primary wrap material. The strap can be configured to be pulled from left to right, e.g., to stretch across the lower abdomen and/or left lower abdominal quadrant from the left side of the patient's body towards right side of the patient's body. This placement of the secondary strap 72 may enable the compression to be easily adjusted as the patient lies on their left side during the procedure. As the strap pulls from left to right across the lower abdomen, additional leverage and compression may be generated by the patient's body when the strap is in place. As the strap stretches from left to right cross the patient's abdomen, the level of compression generated by the device can easily be adjusted during the procedure, e.g., while the patient is lying on their left side.

The secondary strap 72 may attach to the exterior side 78 of the primary wrap 10, with a closing mechanism 74 that allows the strap, e.g., to be fastened to the exterior side 78 of the primary wrap 10. In an aspect, the closing mechanism 74 of the secondary strap 72 may comprise a hook strip 75 on the inside of the secondary strap 72 that is capable of fastening anywhere along the exterior side 78 of the primary wrap 10. In this example, the exterior side 78 of the primary wrap 10 may comprise a hook-compatible material to which the hook strip 75 can be fastened and remain fastened while the secondary strap is stretched and under tension. The secondary strap 72 may be constructed of an elastic or semi-elastic material that is capable of retaining tension when stretched horizontally and fastened to the exterior side 78 of the primary wrap 10 using the closing mechanism 74. The secondary strap 72 may comprise the same or different materials than the materials comprised in the primary wrap 10. The secondary strap 72 may comprise one or more layers of materials. In an example, the secondary strap 72 may comprise an elastic strap the entire length of which equals approximately twice the intended length of the secondary strap 72. As illustrated, the secondary strap may taper along a length of the strap 72. In the example in FIG. 2, the elastic strap may be doubled-over, and both ends may be sewn to the exterior 78 of the primary wrap 10 along the same vertical line 73, creating the horizontal ‘V’ appearance of the secondary strap 72. Doubling-over the material composing the secondary strap can serve to increase the breadth and force generated by the secondary strap, while preventing the incurrence of additional materials costs that would be associated with using a taller, single-layered strap instead. The midpoint of the secondary strap may be positioned approximately over the patient's lower left quadrant once it is stretched and fastened. In some aspects, the secondary strap 72 may be a unitary piece having at least one tapering edge.

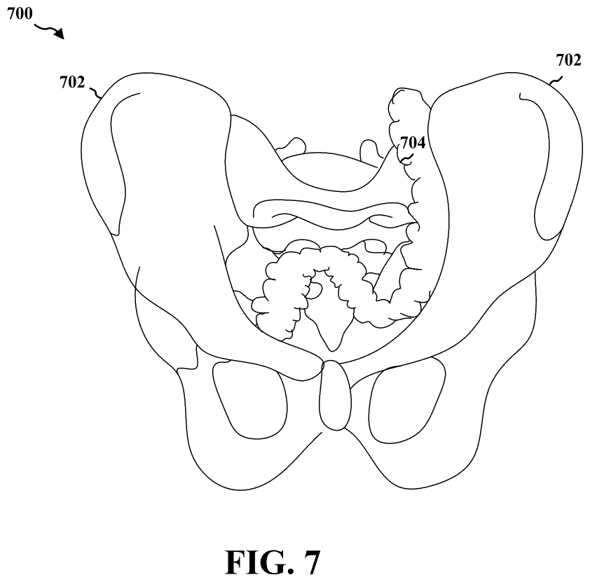

In some aspects, patient anatomy may limit the compression applied by the device. FIG. 7 illustrates an example 700 showing that the iliac crest 702 that can hold the device away from the colon 704 to which compression is intended to be applied. For example, in patients with a low BMI, the iliac crest may extend beyond the abdomen and may hold the primary wrap and secondary strap away from compressing the abdomen.

In order to apply pressure, e.g., compression, to the abdomen, one or more inserts may be placed between at least a portion of the device and the patient's abdomen. In some aspects, the insert may be placed between the patient's abdomen and the primary band, for example. The insert may be configured to communicate compression from the primary band and/or secondary strap to the abdomen of the patient. The insert can be used in combination with the compression device to improve the efficacy of the compression.

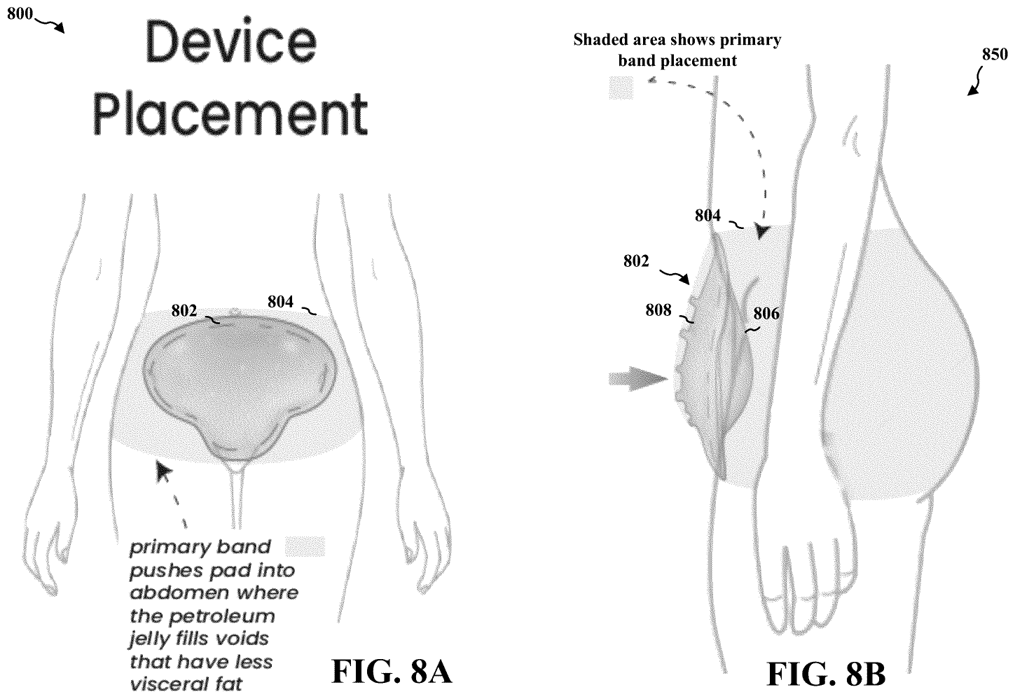

In some aspects, the insert may include a filled pad 802 that is configured to cover an abdominal region of a patient, e.g., as shown in front view 800 in FIG. 8A and side view 850 in FIG. 8B and shaped to extend into the abdominal region. The shaded area 804 around the abdomen and hips shows the placement for a compression device, which may also be referred to as a compression band or compression wrap, such as described in connection with FIGS. 2-6. In some aspects, the pad 802 may be placed on the abdomen of the patient, and the primary wrap and the secondary straps may be connected over the top of the pad. In some aspects, the pad 802 may be applied between the primary wrap and the secondary straps.

FIG. 8A describes that the primary band pushes the pad 802 into the abdomen such that the pad fills any void that may have less visceral fat. This allows the compression of the primary wrap and secondary strap to be communicated from the compression device to the colon, e.g., via the filling provided by the pad 802. The pad 802 helps to fill the area between the abdomen and the band that may be created by the iliac crest in patients having lower BMI. In some aspects, the pad may be filled with a material having a density similar to adipose tissue. The fill may include a viscous material or a gel. In some examples, the viscous material may include a petroleum jelly or a material having a similar density. As another example, the fill may include a ballistics gel. In other aspects, the fill may include a fluidized material comprised of a particulate material that has been modified in some way to take on characteristics of a fluid. Fluidized materials can include compressible and/or non-compressible beads (e.g., which may also be referred to as balls, spheres, spherical components, or rounded components, among other examples), such as polyethylene or polystyrene beads, expanded polyethylene, crosslinked expanded polyethylene, polypropylene pellets, open and closed cell foams, microspheres, and/or encapsulated phase changing materials. The beads can be hard shelled and/or flexible. In some aspects, the fluidized material may comprise a porous foam substance with interstitial pockets of air or other fluids. In some aspects, the fluidized material may include polyurethane foam, with or without other fluids. Other fluidized materials may comprise a mixture of microspheres and lubricants. In some aspects, the microspheres may include, or may consist of, hollow or gas filled bubbles or beads. In some aspects the microspheres may be comprised of silicone, glass, ceramic, steel, or aluminum. In some aspects, the fluidized material may include ballistics gel, bentonite clay, vegetable glycerin, xanthan gum, beeswax, gelatin, aloe vera gel, PVC with non-toxic plasticizers, PVC gel, water, carboxymethyl cellulose (CMC), hydroxyethyl cellulose, guar gum, agar, methylcellulose, sodium alginate, cornstarch, tapioca starch, and/or pectin. In some aspects, the fluidized material may contain, or include, gases such as room air, oxygen, carbon dioxide, helium, hydrogen, or nitrogen. In some aspects, the fluidized material may have properties that render the insert moldable prior to being applied against the patient. In such aspects, the fluidized material could include, or could consist of, a mixture of microspheres and a liquid oil plasticizer with an elastomeric polymer dissolved within the oil plasticizer. In such aspects, the materials enable the endoscopy provider to mold the insert to the desired shape before applying it against the patient and compressing it into the abdomen using the compression device. In other aspects, the insert may contain fixed form components surrounded by fluidized materials and enveloped in the shell (e.g., housing or main body) of the pad. In one example, this could include shaped gel or foam objects surrounded by a viscous fluidized material. In some aspects, the fill may have biomimetic properties to human visceral adipose tissue. For example, human visceral adipose tissue may have a density of approximately 0.9 g/cm³. The fill may also have a density of 0.9 g/cm³ or may have a density in a range of 0.89-0.91 g/cm³, in a range of 0.87-0.93 g/cm³, in a range of 0.85-0.95 g/cm³, or in a range of 0.75-1.5 g/cm³, for example.

In some aspects, portions of the shell (e.g., housing or main body) of the pad and the fluidized media may contain silicone, nitrile, polyurethane elastomers, thermoplastic elastomers (TPE), polyisoprene, polyvinyl chloride (PVC) with non-toxic plasticizers, ethylene propylene diene monomers, stryrene-ethylene-butylene-stryene (SEBS), polyurethane foam, thermoplastic polyurethane foam (TPU), and/or ethyl-vinyl acetate (EVA).

In some aspects, the insert/pad may include an independent, sealed interstitial space, e.g., a space completely encapsulated by one or more fluidized materials within the insert, and containing chemicals or components that when activated produce a pressurized chamber that assists in delivering compression to patient's abdomen. In other embodiments the insert may contain chemicals that can be activated by the injection of water, air, saline, oxygen, carbon dioxide, or other materials into the insert. The chemicals may create fluidized materials with specific properties upon the introduction of chemicals into the insert. Differing characteristics of the fluidized material may be achieved through the injection of differing volumes of the same or different chemicals one or more times. The shell of the insert may be comprised of a self-healing material that does not weaken or leak following puncture with a bore, needle, or other instrument. In some aspects, the insert may contain one or more spouts or ports designed to accommodate infusion and withdrawal of materials. Similarly, in other aspects, the insert may comprise one or more bladders embedded amidst the fluidized material that may be inflated and deflated via ports extending to and from the bladder through the fluidized material and the shell.

The pad 802 may include an outer housing or shell. The shell may be comprised of two portions with one firmer, fixed or semi-fixed form portion on the side of the insert adjacent to the compression band, and a second softer side designed to be place against and compressed into the patients' abdomen.

The outer housing may include at least a portion that is flexible so that the pad and inner fill can conform to the abdominal region of the patient. In some aspects, a first side 806 of the pad, e.g., a side that is positioned toward the abdomen of the patient, may include a first material, and the opposite side (second side 808) may include a second material. This enables the side 806 to have a material with a lower hardness than the second side 808. The more flexible material placed toward the abdomen of the patient enables the pad to adjust to the anatomy of the patient to fill the area of individual patients. The added hardness of the second side 808 opposite the patient provides added structural support for the pad so that it retains the overall intended shape even when compression is applied by the outer compression device. As an example, the first side 806 may have a shore hardness of 00-10. It may comprise a 00-10 silicon material, which has more elasticity than the second side 808. In some examples, the second side 808 may include a material with a shore hardness of 10 Å. In some aspects, both sides of the pad may comprise a silicon material. The outer housing of the pad 802 may also comprise other biocompatible materials. At least a portion of exterior shell of the insert may also be comprised of soft, flexible biocompatible material such as silicone or polyurethane given that it will be directly contacting the patient's skin. In some aspects, this could comprise a vulcanized silicone elastomer. Some aspects may employ a soft, flexible film shell to minimize shear forces against the patient's skin when the insert is compressed into the abdomen.

The shell may include seals, bondings, or closures that are soft, pliable, and/or in certain embodiments elastic. These edges may be configured to avoid being sharp or hard, as this may increase a risk of patient injury when the insert is compressed into the abdomen. The edges, seals, or closure aspects of the shell may render the pad shaped, semi-shaped, or amorphous. In certain embodiments, the tendency for the insert to retain its shape can may be controlled by a relationship between the volume of filler relative to the entirety of interstitial space enclosed by the shell (e.g., housing of the pad). In some aspects, the insert may contain a fluidized material with responsive components and characteristics that change in response to certain forces and/or stresses. For example, the fluidized material may be fluid, malleable, and amorphous at rest, allowing the insert to be easily adapted to the unique anatomy of individual patients. Yet in response to the compressive force delivered by the compression device, the fluidized material becomes firmer, enabling the insert to achieve efficient translation of abdominal compression into the patient's abdomen.

In some aspects, the pad may include a pressure reactive material, such as a material that becomes harder when pressure is applied or a material that provides a visual effect (e.g., changing color, etc.) when pressure is applied.

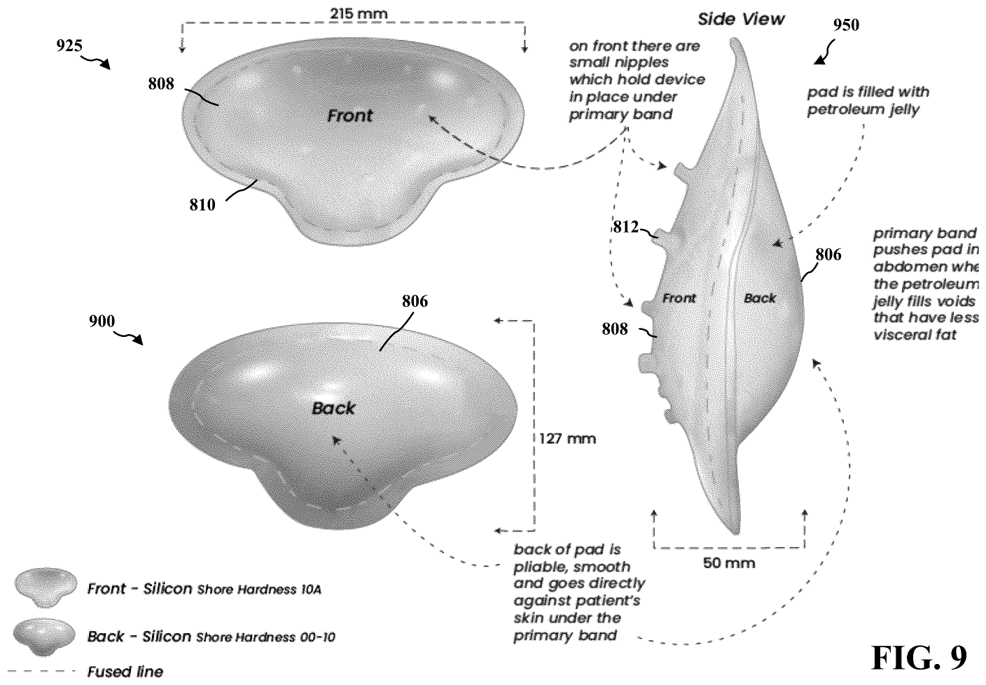

FIG. 9 illustrates an example view 900 from the first side 806, a view 925 from the second side 808, and a side view 950 of the pad 802. FIG. 9 shows a dashed line 810 where the pad may include a fused portion that fuses or permanently connects the two sides of the pad 802. The portion of the pad extending beyond the line 810 may be sized to avoid a rupture of the pad when pressure is applied by a compression band. The side 808 may include protrusions 812 to provide grip or maintain a position of the pad once the compression device is applied over the pad. The protrusions may also have a pointed end. Other mechanisms may also be provided at the side 808 to help maintain the placement of the pad relative to the compression device. For example, a hook material may be included at the side 808, or an adhesive may be included at the side 808. The side 806 may have a smooth surface.

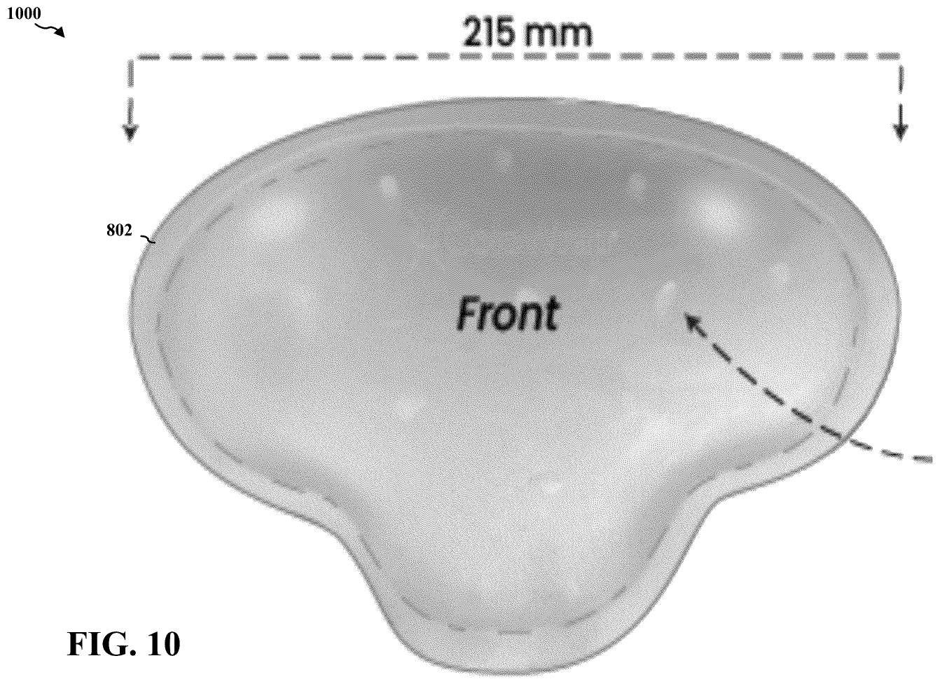

FIG. 10 illustrates an example view 1000 from the second side 808 of the pad 802, e.g., the side to be positioned away from the patient, e.g., toward the compression device. FIG. 10 illustrates an example length of an upper portion of the pad as 215 mm. This is merely one example. In some aspects, different sizes of pads may be provided so that a user can select one based on the anatomy of a patient. The upper portion of the pad may be sized to fit approximately between the iliac crest area of the patient's hips. The lower portion of the pad may taper.

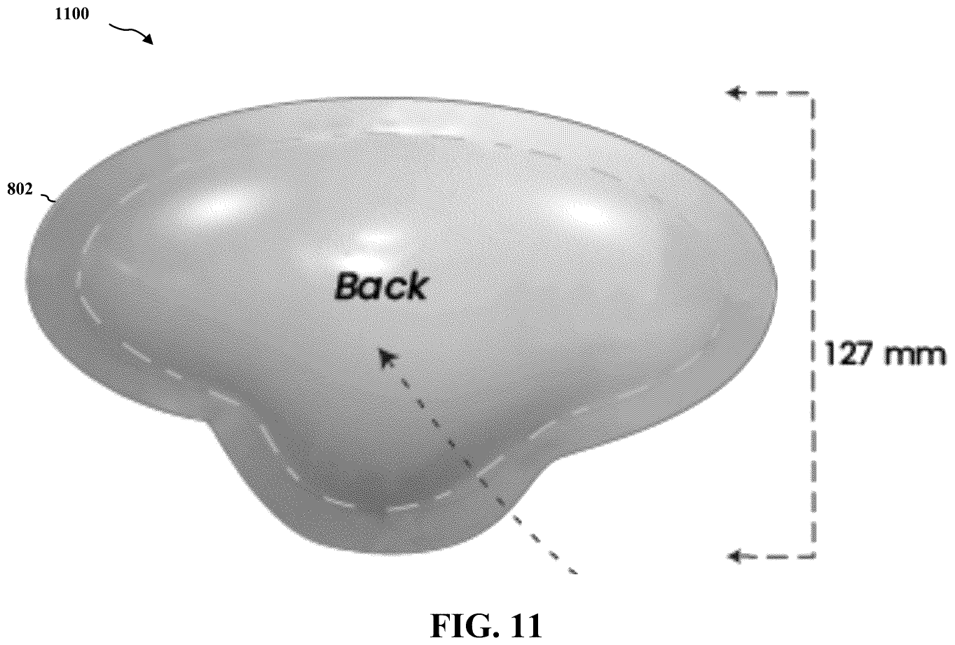

FIG. 11 illustrates a side view 1100 of the pad 802 from the first side, e.g., to be positioned toward the abdomen of the patient. FIG. 11 illustrates an example length of 127 mm, e.g., which may be configured to span the height of a lower abdomen of a patient. As with the length, this is merely one example of a height for the pad, and different sizes of pads may be provided so that a user can select one based on the anatomy of a patient.

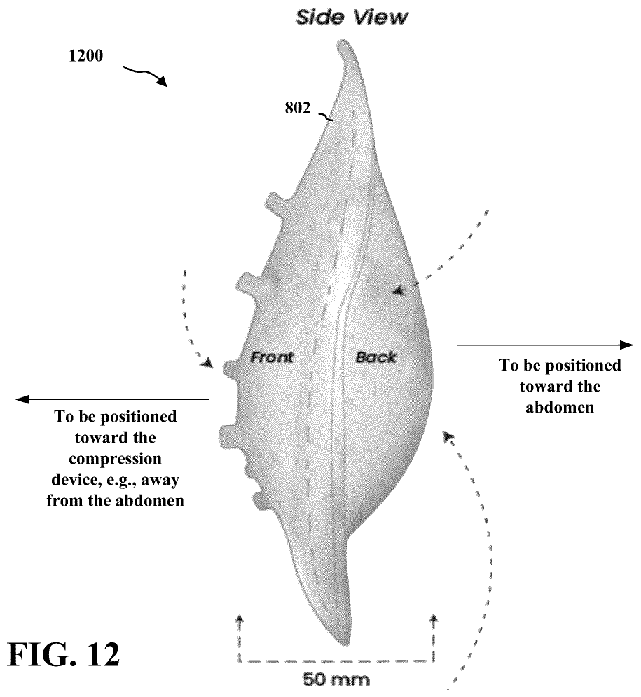

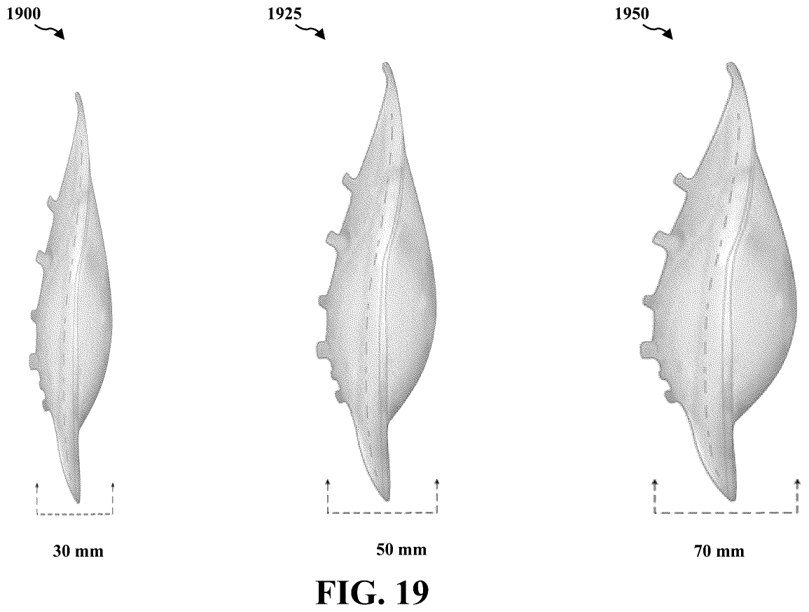

FIG. 12 illustrates a side view 1200 of the pad 802 and shows an example width of 50 mm. As with the height and length, this is merely one example of a width for the pad, and different sizes of pads may be provided so that a user can select one based on the anatomy of a patient. FIG. 19 illustrates different example widths, e.g., shown at 1900, 1925, and 1950, for a pad 802. FIG. 12 shows that the width of the pad changes along the height of the pad 802. For example, a middle region has a greatest width, and the upper and lower portions taper.

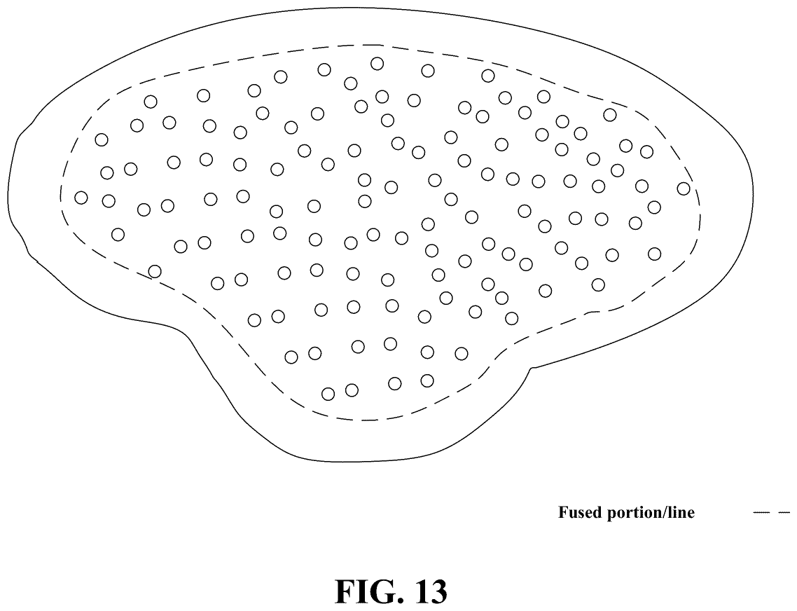

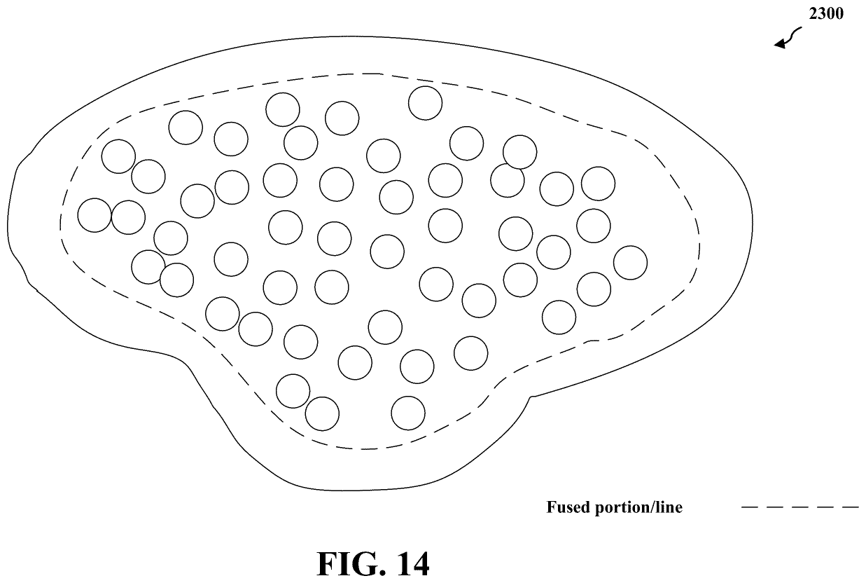

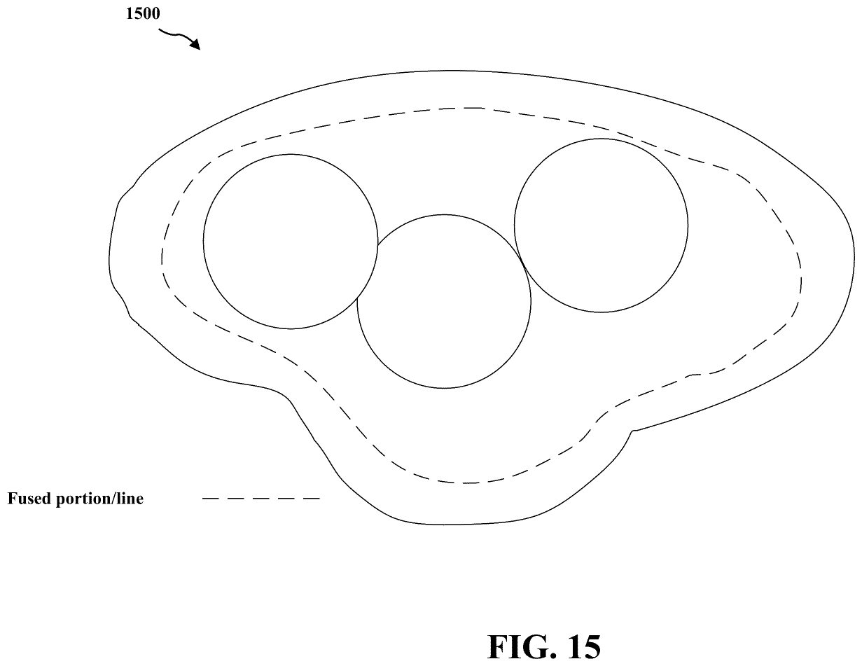

The viscous filler may also include one or more moveable items provided within the filler. As an example, the filler may include a plurality of spherical balls or beads suspended within the viscous fill material. As shown in FIGS. 13, 14, and 15, the spherical balls may have different sizes. Smaller spherical balls may be added to provide added weighting or shaping. For example, the weighted spherical balls may have a diameter between 4-6 mm, in some examples. In other examples, the spherical balls may have a structure more similar to marbles, e.g., a diameter of 14-18 mm. In other examples, e.g., such as shown in the example 1500 in FIG. 15, the spherical balls may have a diameter of 1.5-1.7 inches. In some aspects, the added components within the filler may provide a moldable structure to the pad to enable the filler to be manipulated to provide added support in particular areas of the abdomen. In some aspects, the spherical balls in FIG. 15 may be moved or manipulated within the pad 802 to provide a firmer counterpressure for an endoscope during movement within a colon.

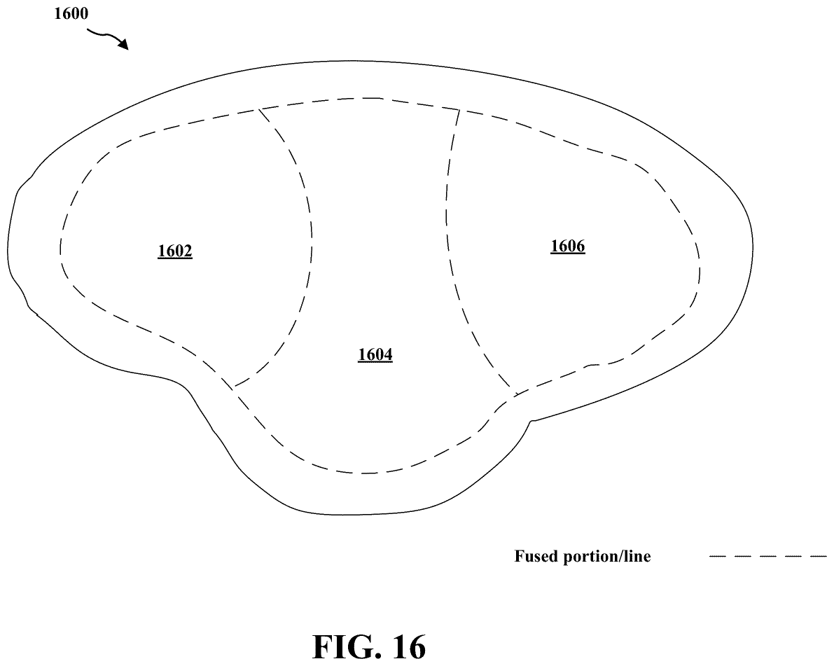

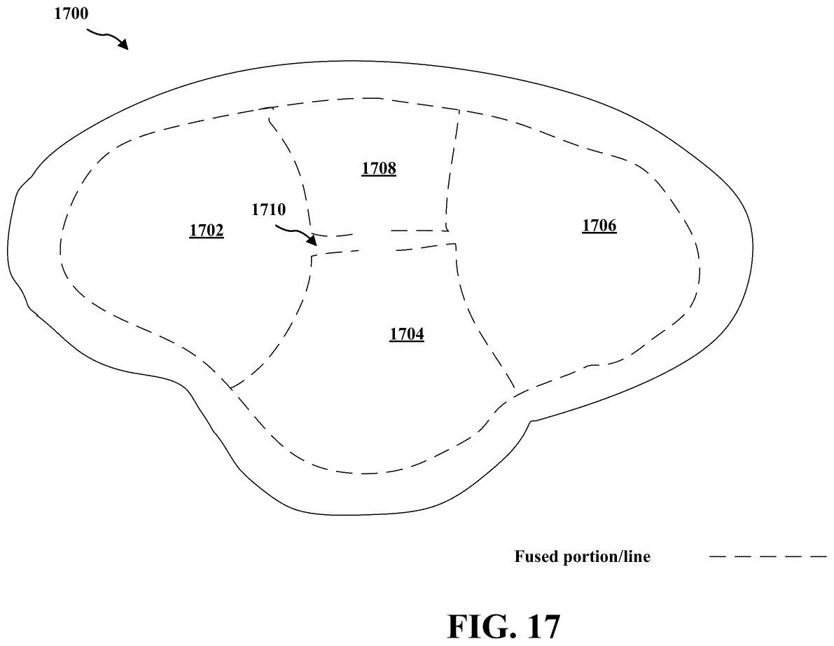

FIG. 16 illustrates an example pad 1600 having multiple compartments (1602, 1604, 1606), each compartment being filled with the viscous filler material or fluidized material. The compartments may have different shapes and placements than those illustrated in FIG. 16. FIG. 16 illustrates an example in which the compartments are fully enclosed and separate. FIG. 17 illustrates an example 1700 having multiple connected compartments (1702, 1704, 1706, 1708) that are connected via one or more channels 1710. Compartments may also comprise layers of similar or differing fluidized materials or other components stacked (between the compression device and the abdomen of the patient) within the shell.

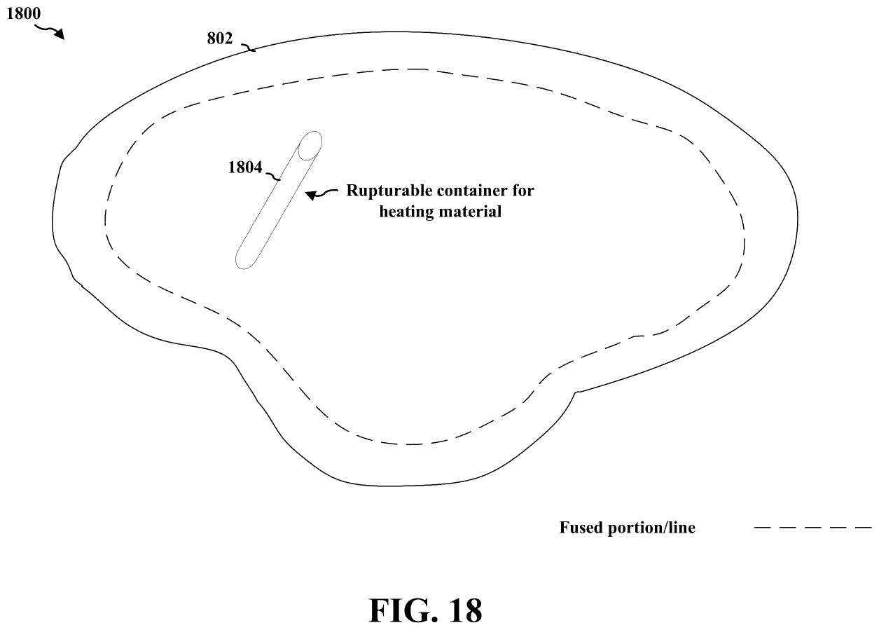

In some aspects, the pad may include a heating component so that the pad may be warmed prior to placement on the patient's abdomen. As one example 1800, shown in FIG. 18, the pad 802 may include a rupturable container 1804 that can be broken or cracked by a user to release a chemical that warms the filling. In some aspects, the warmth of the pad may increase the comfort of the patient. The warmth may help to prevent spasms of the colon during the endoscopy procedure.

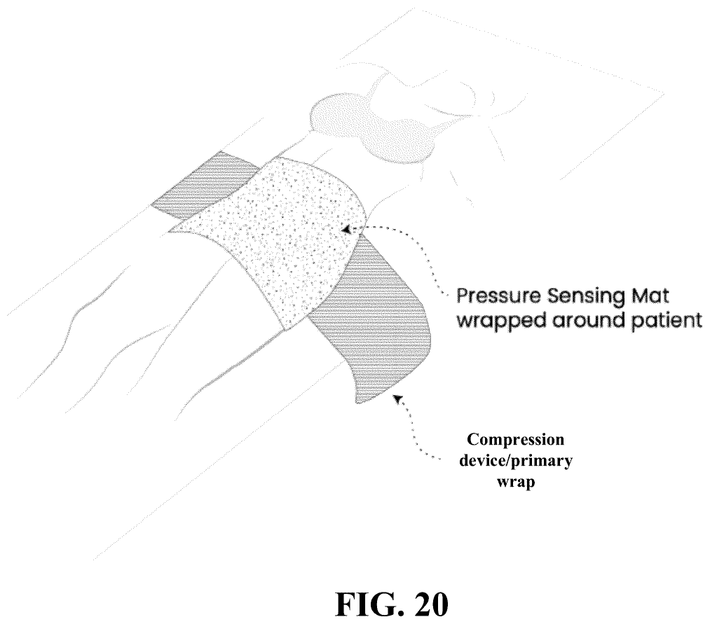

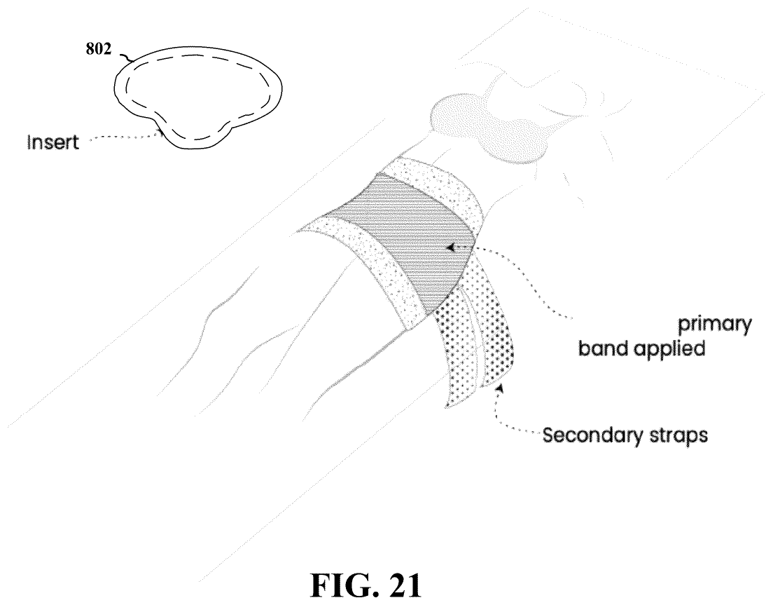

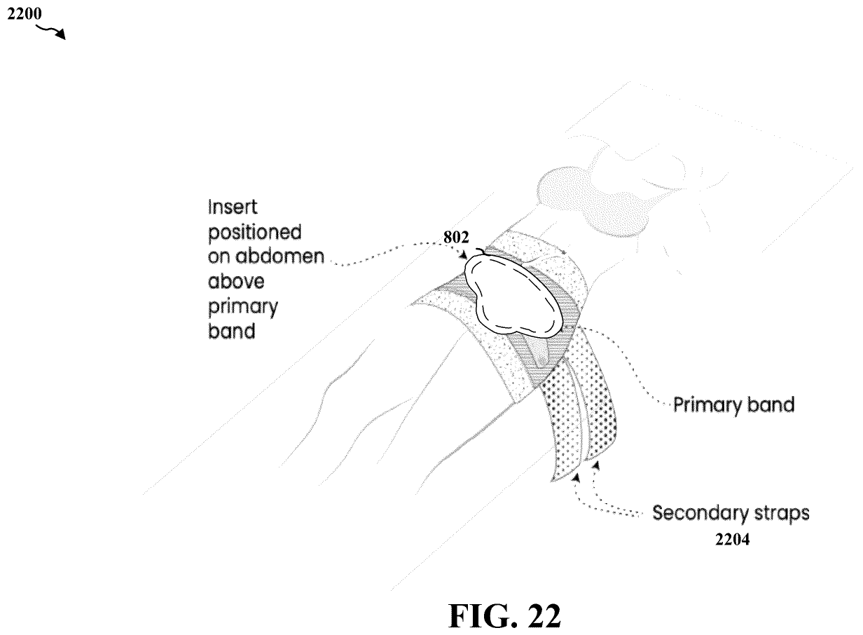

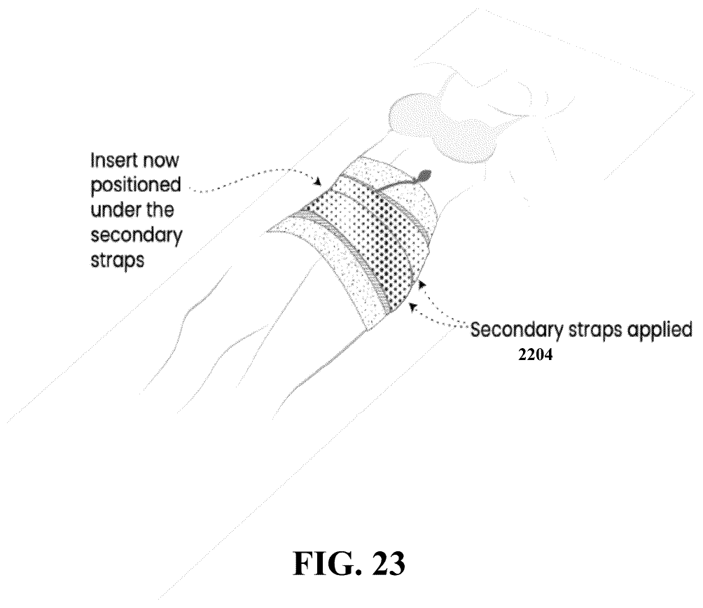

FIGS. 20-23 illustrate an example placement of the insert together with a compression device. FIG. 20 illustrates that a pressure sensitive mat may be wrapped around a patient prior to application of the insert and compression device. The compression device may include any of the aspects described in connection with FIGS. 2-6. FIG. 21 shows the primary band wrapped and secured around the abdomen of the patient. In some aspects, the pad 802 may be placed between the primary band and the secondary straps. FIG. 22 illustrates a view 2200 showing the insert positioned on the abdomen of the patient prior to application of the secondary straps 2204, according to this example placement. In other aspects, the pad 802 may be placed on the abdomen of the patient prior to application of the primary wrap. FIG. 23 illustrates a view 2300 in which the secondary straps 2204 are secured over the pad. In some aspects, the insert may be applied to the abdomen of the patient prior to application of the primary wrap.

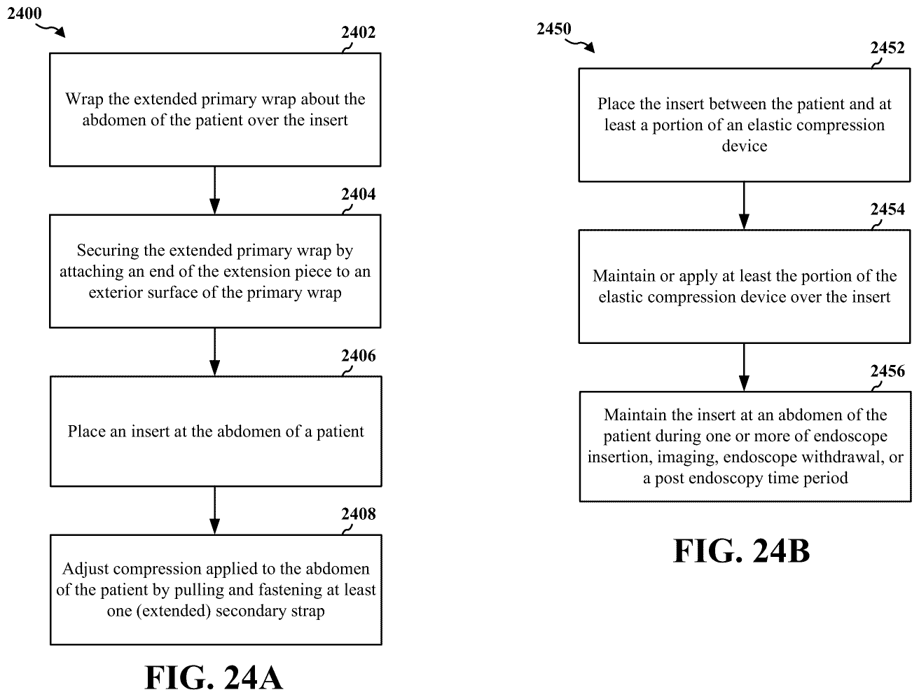

FIGS. 24A and 24B illustrate a flowchart 2400 and 2450 aspects of methods of applying an insert with an endoscopy support device. The method illustrated in FIG. 24B may further include any of the aspects described in connection with the flowchart in FIG. 24A, and the method illustrated in FIG. 24A may further include any of the aspects described in connection with FIG. 24B, for example. The method may provide added, external support and guidance for movement of an endoscope during an endoscopy procedure.

FIG. 24A illustrates a flowchart 2400 of a method of applying an insert with an endoscopy support device. At 2402, a user wraps a primary wrap about the abdomen of the patient, e.g., as described in connection with any of FIG. 2-7 or 21. The user secures the primary wrap at 2404, by attaching an end of the extension piece to an exterior surface of the primary wrap. At 2406, the user places an insert at the abdomen of a patient, e.g., as shown in any of FIGS. 8A, 8B, and/or 21. The insert may include any of the aspects described in connection with FIGS. 8A-19, for example. At 2408, the user adjusts the compression applied to the abdomen of the patient by pulling and fastening at least one secondary strap, e.g., as illustrated in any of FIG. 4 or 23. The user may readjust the pressure applied to the abdomen by adjusting one or more secondary strap.

FIG. 24B illustrates an example of a method of applying an insert at a patient in connection with an endoscopy procedure. At 2452, the method includes placing the insert between the patient and at least a portion of an elastic compression device, wherein the insert includes: an outer housing having at least a portion comprising a flexible, pliable material; and an inner material within the outer housing, wherein the inner material has at least one property related to adipose tissue. FIGS. 8A, 8B, 20-23 illustrate examples aspects of insert placement

At 2454, the method includes maintaining or applying at least the portion of the elastic compression device over the insert. FIG. 23 illustrates an example of applying the secondary straps and a compression device over the insert.

At 2456, the method includes maintaining the insert at an abdomen of the patient during one or more of endoscope insertion, imaging, endoscope withdrawal, or a post endoscopy time period.

In some aspects, the method further includes wrapping a primary wrap of the elastic compression device around the abdomen of the patient before placing the insert on an exterior side of the primary wrap, wherein the insert is placed between the primary wrap and at least one secondary strap of the elastic compression device. FIGS. 20 and 21 illustrate examples of applying a primary wrap.

In some aspects, the method further includes adjusting compression applied to the abdomen of the patient by fastening the at least one secondary strap of the elastic compression device over the insert, e.g., such as shown in connection with any of FIGS. 3A, 4, 6, and/or 23.

In some aspects, the insert may be positioned between the abdomen of the patient and a primary wrap of the elastic compression device.

In some aspects, the method further includes adjusting compression delivered to the abdomen via the insert based on one or more of: adjustment of a primary band of the elastic compression device, adjustment of at least one secondary strap of the elastic compression device, adjustment of insert placement, or adjustment of a moveable component of the insert.

In some aspects, the method further includes iterative application and removal of the insert multiple times during an endoscopic procedure.

In some aspects, the method further includes placing a pressure sensor between the abdomen of the patient and the elastic compression device. FIG. 20 illustrates an example of a sensor.

In some aspects, the method further includes inserting at least one of a fluid or a gas into an interior of the outer housing to generate the inner material as a viscous material or change a property of the insert.

In some aspects, the method further includes manipulating one or more moveable components within the outer housing to adjust compression to the abdomen of the patient.

In some aspects, the method further includes manipulating the inner material of the insert to form a moldable structure to assist in targeted compression at the abdomen.

In some aspects, the method further includes rupturing a container within the outer housing to cause a reaction that warms the insert.

The insert may be used, or applied, in any of various potential ways. In some aspects, the use of the insert may be particular to a particular patient and/or a particular part of the procedure. For example the insert may be applied pre-procedurally, intra-procedurally, during either an endoscope insertion phase, an imaging phase, and/or an endoscope withdrawal phase. The insert may be applied, or adjusted, at any point of the endoscopy procedure. Adjustments to compression may be delivered via the insert through adjustment of the compression device (e.g., the primary band and/or secondary straps) and/or adjustment of any insert component or feature designed to offer adjustability (e.g. placement and/or inflation or deflation of an air bladder, chemical activation, infusion of air/water/other in differing volumes and/or to different chambers, among other examples). In some aspects, the method may include iterative application and full removal of the insert at various times during the endoscopy procedure (e.g., application, removal, application, removal, application, removal, and so forth). As an example, the insert may be applied while the endoscope is being advance, and then removed. The insert may be reapplied when the endoscope is advanced again, and/or when the endoscope is withdrawn. As another example, the insert may be applied and removed to obtain various images or views of the colon.

In some aspects, maintaining the compression applied by the device during the withdrawal phase of the procedure and while imaging is performed may help improve the detection of adenoma. The device may be maintained on the patient to reduce the common post-procedure complications of bloating and abdominal pain caused by bloating. Otherwise known as gaseous distention, bloating occurs following endoscopy procedures because physicians often use compressed air or carbon dioxide to insufflate parts of the bowel that are difficult to see and examine. The gas opens up the area to allow for a more complete visualization, enhancing the efficacy of the procedure. However, the gas also remains in the patient until it is either absorbed or expelled. Expulsion is the primary gas removal mechanism as absorption is a very inefficient process. Gaseous distention is a primary post-procedure complication and a frequent complaint from patients. However, when the wrap described herein remains in place after the procedure, the lower abdominal compression generated by the device allows the bowel to more rapidly evacuate trapped by directing excess gas towards the rectum. As a result, the severity and duration of post-procedure bloating and associated abdominal pain may be reduced.

In some aspects, an apparatus for placement at an abdomen of a patient in connection with an endoscopy procedure, comprising: an outer housing having at least a portion comprising a flexible, pliable material; and an inner material within the outer housing, wherein the inner material has at least one property related to adipose tissue. The apparatus may be referred to herein as an insert, a viscous insert, a malleable insert, a pad, among other examples. FIGS. 8A-23 illustrate example aspects of such an apparatus.

In some aspects, the outer housing includes a wider portion configured for the placement at the abdomen of the patient between iliac crests of hips of the patient, wherein the wider portion includes a first edge and a second edge, and wherein the first edge and the second edge of the wider portion each taper to a narrower portion configured for the placement at a lower abdominal region below the iliac crests. FIG. 8A illustrates an example of a shape of an example apparatus (e.g., 802).

In some aspects, the outer housing includes a first side including a first material, and a second side including a second material and configured for the placement toward the abdomen of the patient, wherein the first material is different than the second material, and wherein the second material has a lower hardness than the first material. FIG. 9 illustrates an example of a device having different materials for a front and back.

In some aspects, the apparatus includes one or more protrusions extending from the first side, a hook material at the first side, or an adhesive positioned at the first side. FIGS. 8B and 9 illustrates examples of protrusions.

In some aspects, the at least one property of the inner material includes a density within a range of 0.75-1.5 g/cm³. In some aspects, the at least one property of the inner material includes a density within a range of 0.85-0.95 g/cm³.

In some aspects, the inner material includes one or more of a viscous material, a gel, or a fluidized material. FIG. 8A-22 illustrate various examples of an apparatus having an inner material that is viscous or malleable. In some aspects, the inner material includes a first material that becomes viscous or malleable when combined with a fluid. In some aspects, to receive the fluid, the outer housing comprises one or more of: a self-healing material, a spout, or a port.

In some aspects, the apparatus includes one or more moveable components within the outer housing. FIGS. 13-15 illustrate examples of moveable components within the outer housing. In some aspects, the one or more moveable components includes a plurality of spherical components that are freely moveable within the inner material. In some aspects, the one or more moveable components includes a porous foam. In some aspects, the apparatus further includes a gas within the outer housing. For example, one or more of the compartments (e.g., 1602, 1604, or 1606) shown in FIG. 16 may include a gas. In some aspects, one of more of the compartments may form, or include an inflatable bladder.

In some aspects, the inner material forms a moldable structure within the outer housing. For example, a shape of the moldable structure may be maintained based on a relationship between a volume of the inner material relative to an interstitial space enclosed by the outer housing.

In some aspects, the one or more of the outer housing or the inner material may include a pressure reactive material.

In some aspects, the outer housing may include multiple compartments, each compartment filled with one or more inner materials. FIGS. 16 and 17 illustrate various example aspects of an apparatus including compartments. The multiple compartments may be fully enclosed and separate. The multiple compartments may be multiple compartments are connected via one or more channels. Each of the multiple compartments may include a same filler materials. In some aspects, the multiple compartments may include different filler material.

In some aspects, the apparatus may further include a heating component within the outer housing. As an example, the heating component may include a rupturable container comprising a chemical that reacts when ruptured to warm the apparatus. FIG. 18 illustrates an example of a heating component.

Example aspects of the present invention have now been described in accordance with the above advantages. It will be appreciated that these examples are merely illustrative of aspects of the present invention. Many variations and modifications will be apparent to those skilled in the art.

It is understood that the specific order or hierarchy of steps in the processes disclosed is an illustration of exemplary approaches. Based upon design preferences, it is understood that the specific order or hierarchy of steps in the processes may be rearranged. Further, some steps may be combined or omitted. The accompanying method claims present elements of the various steps in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects.” Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

The following aspects are illustrative only and may be combined with other aspects or teachings described herein, without limitation.

Aspect 1 is an apparatus, e.g., which may be referred to as an insert, for placement at an abdomen of a patient in connection with an endoscopy procedure, comprising: an outer housing having at least a portion comprising a flexible, pliable material; and an inner material within the outer housing, wherein the inner material has at least one property related to adipose tissue.

In aspect 2, the apparatus of aspect 1 further includes that the outer housing includes a wider portion configured for the placement at the abdomen of the patient between iliac crests of hips of the patient, wherein the wider portion includes a first edge and a second edge, and wherein the first edge and the second edge of the wider portion each taper to a narrower portion configured for the placement at a lower abdominal region below the iliac crests.

In aspect 3, the apparatus of aspect 1 or aspect 2 further includes that the outer housing includes a first side including a first material, and a second side including a second material and configured for the placement toward the abdomen of the patient, wherein the first material is different than the second material, and wherein the second material has a lower hardness than the first material.

In aspect 4, the apparatus of any of aspects 1 to 3 further includes one or more protrusions extending from the first side, a hook material at the first side, or an adhesive positioned at the first side.

In aspect 5, the apparatus of any of aspects 1 to 4 further includes that the at least one property of the inner material includes a density within a range of 0.75-1.5 g/cm³.

In aspect 6, the apparatus of any of aspects 1 to 4 further includes that the at least one property of the inner material includes a density within a range of 0.85-0.95 g/cm³.

In aspect 7, the apparatus of any of aspects 1 to 6 further includes that the inner material includes one or more of a viscous material, a gel, or a fluidized material.

In aspect 8, the apparatus of any of aspects 1 to 6 further includes that the inner material includes a first material that becomes viscous or malleable when combined with a fluid.

In aspect 9, the apparatus of aspect 8 further includes that to receive the fluid, the outer housing comprises one or more of: a self-healing material, a spout, or a port.

In aspect 10, the apparatus of any of aspects 1 to 9 further includes one or more moveable components within the outer housing.

In aspect 11, the apparatus of aspect 10 further includes that the one or more moveable components includes a plurality of spherical components that are freely moveable within the inner material.

In aspect 12, the apparatus of aspect 10 or 11 further includes that the one or more moveable components includes a porous foam.

In aspect 13, the apparatus of any of aspects 10 to 12 further includes that a gas within the outer housing.

In aspect 14, the apparatus of any of aspects 1 to 13 further includes that the apparatus further includes an inflatable bladder.

In aspect 15, the apparatus of any of aspects 1 to 14 further includes that the inner material forms a moldable structure within the outer housing.

In aspect 16, the apparatus of aspect 15 further includes that a shape of the moldable structure is maintained based on a relationship between a volume of the inner material relative to an interstitial space enclosed by the outer housing.

In aspect 17, the apparatus of any of aspects 1 to 16 further includes that one or more of the outer housing or the inner material includes a pressure reactive material.

In aspect 18, the apparatus of any of aspects 1 to 17 further includes that the outer housing includes multiple compartments, each compartment filled with one or more inner materials.

In aspect 19, the apparatus aspect 18 further includes that the multiple compartments are fully enclosed and separate.

In aspect 20, the apparatus aspect 18 further includes that the multiple compartments are connected via one or more channels.

In aspect 21, the apparatus of any of aspects 18 to 20 further includes that each of the multiple compartments includes a same filler materials.

In aspect 22, the apparatus of any of aspects 18 to 20 further includes that the multiple compartments include different filler material.

In aspect 23, the apparatus of any of aspects 1 to 22 further includes that a heating component within the outer housing.

In aspect 24, the apparatus of aspect 23 further includes that the heating component includes a rupturable container comprising a chemical that reacts when ruptured to warm the apparatus.

Aspect 25 is a method of applying an insert at a patient in connection with an endoscopy procedure, the method comprising: placing the insert between the patient and at least a portion of an elastic compression device, wherein the insert includes: an outer housing having at least a portion comprising a flexible, pliable material; and an inner material within the outer housing, wherein the inner material has at least one property related to adipose tissue; maintaining or applying at least the portion of the elastic compression device over the insert; and maintaining the insert at an abdomen of the patient during one or more of endoscope insertion, imaging, endoscope withdrawal, or a post endoscopy time period.

In aspect 26, the method of aspect 25 further includes wrapping a primary wrap of the elastic compression device around the abdomen of the patient before placing the insert on an exterior side of the primary wrap, wherein the insert is placed between the primary wrap and at least one secondary strap of the elastic compression device.

In aspect 27, the method of aspect 25 or 26 further includes: adjusting compression applied to the abdomen of the patient by fastening the at least one secondary strap of the elastic compression device over the insert.

In aspect 28, the method of any of aspects 25 or 27 further includes that the insert is positioned between the abdomen of the patient and a primary wrap of the elastic compression device.

In aspect 29, the method of any of aspects 25 to 28 further includes adjusting compression delivered to the abdomen via the insert based on one or more of: adjustment of a primary band of the elastic compression device, adjustment of at least one secondary strap of the elastic compression device, adjustment of insert placement, or adjustment of a moveable component of the insert.

In aspect 30, the method of any of aspects 25 to 29 further includes iterative application and removal of the insert multiple times during an endoscopic procedure.

In aspect 31, the method of any of aspects 25 to 30 further includes placing a pressure sensor between the abdomen of the patient and the elastic compression device.

In aspect 32, the method of any of aspects 25 to 31 further includes that the outer housing includes a wider portion configured for placement at the abdomen of the patient between iliac crests of hips of the patient, wherein the wider portion includes a first edge and a second edge, and wherein the first edge and the second edge of the wider portion taper to a narrower portion configured for the placement at a lower abdominal region below the iliac crests.

In aspect 33, the method of any of aspects 25 to 32 further includes that the outer housing includes a first side including a first material, and a second side including a second material and configured for placement toward the abdomen of the patient, wherein the first material is different than the second material, and wherein the second material has a lower hardness than the first material.

In aspect 34, the method of any of aspects 25 to 33 further includes that the at least one property of the inner material includes a density within a range of 0.75-1.5 g/cm³.

In aspect 35, the method of any of aspects 25 to 33 further includes that the inner material includes one or more of a viscous material, a gel, or a fluidized material.

In aspect 36, the method of any of aspects 25 to 35 further includes inserting at least one of a fluid or a gas into an interior of the outer housing to generate the inner material as a viscous material or change a property of the insert.

In aspect 37, the method of any of aspects 25 to 36 further includes manipulating one or more moveable components within the outer housing to adjust compression to the abdomen of the patient.

In aspect 38, the method of any of aspects 25 to 36 further includes manipulating the inner material of the insert to form a moldable structure to assist in targeted compression at the abdomen.

In aspect 39, the method of any of aspects 25 to 38 further includes that the outer housing includes multiple compartments, each compartment filled with one or more inner materials.

In aspect 40, the method of any of aspects 25 to 39 further includes rupturing a container within the outer housing to cause a reaction that warms the insert.

1. An apparatus for placement at an abdomen of a patient in connection with an endoscopy procedure, comprising:
an outer housing having at least a portion comprising a flexible, pliable material; and
an inner material within the outer housing, wherein the inner material has at least one property related to adipose tissue.

2. The apparatus of claim 1, wherein the outer housing includes a wider portion configured for the placement at the abdomen of the patient between iliac crests of hips of the patient, wherein the wider portion includes a first edge and a second edge, and wherein the first edge and the second edge of the wider portion each taper to a narrower portion configured for the placement at a lower abdominal region below the iliac crests.

3. The apparatus of claim 1, wherein the outer housing includes a first side including a first material, and a second side including a second material and configured for the placement toward the abdomen of the patient, wherein the first material is different than the second material, and wherein the second material has a lower hardness than the first material.

4. The apparatus of claim 3, further comprising:
one or more protrusions extending from the first side, a hook material at the first side, or an adhesive positioned at the first side.

5. The apparatus of claim 1, wherein the at least one property of the inner material includes a density within a range of 0.75-1.5 g/cm³.

6. The apparatus of claim 1, wherein the at least one property of the inner material includes a density within a range of 0.85-0.95 g/cm³.

7. The apparatus of claim 1, wherein the inner material includes one or more of a viscous material, a gel, or a fluidized material.

8. The apparatus of claim 1, wherein the inner material includes a first material that becomes viscous or malleable when combined with a fluid.

9. The apparatus of claim 8, wherein to receive the fluid, the outer housing comprises one or more of:
a self-healing material,
a spout, or
a port.

10. The apparatus of claim 1, further comprising:
one or more moveable components within the outer housing.

11. The apparatus of claim 10, wherein the one or more moveable components includes a plurality of spherical components that are freely moveable within the inner material.

12. The apparatus of claim 10, wherein the one or more moveable components includes a porous foam.

13. The apparatus of claim 1, further comprising a gas within the outer housing.

14. The apparatus of claim 13, wherein the apparatus further includes an inflatable bladder.

15. The apparatus of claim 1, wherein the inner material forms a moldable structure within the outer housing.

16. The apparatus of claim 15, wherein a shape of the moldable structure is maintained based on a relationship between a volume of the inner material relative to an interstitial space enclosed by the outer housing.

17. The apparatus of claim 1, wherein one or more of the outer housing or the inner material includes a pressure reactive material.

18. The apparatus of claim 1, wherein the outer housing includes multiple compartments, each compartment filled with one or more inner materials.

19. The apparatus of claim 18, wherein the multiple compartments are fully enclosed and separate.

20. The apparatus of claim 18, wherein the multiple compartments are connected via one or more channels.

21. The apparatus of claim 18, wherein each of the multiple compartments includes a same filler materials.

22. The apparatus of claim 18, wherein the multiple compartments include different filler material.

23. The apparatus of claim 1, further comprising:
a heating component within the outer housing.

24. The apparatus of claim 23, wherein the heating component includes a rupturable container comprising a chemical that reacts when ruptured to warm the apparatus.

25. A method of applying an insert at a patient in connection with an endoscopy procedure, the method comprising:
placing the insert between the patient and at least a portion of an elastic compression device, wherein the insert includes:
an outer housing having at least a portion comprising a flexible, pliable material; and
an inner material within the outer housing, wherein the inner material has at least one property related to adipose tissue;
maintaining or applying at least the portion of the elastic compression device over the insert; and
maintaining the insert at an abdomen of the patient during one or more of endoscope insertion, imaging, endoscope withdrawal, or a post endoscopy time period.

26. The method of claim 25, further comprising:
wrapping a primary wrap of the elastic compression device around the abdomen of the patient before placing the insert on an exterior side of the primary wrap, wherein the insert is placed between the primary wrap and at least one secondary strap of the elastic compression device.

27. The method of claim 26, further comprising:
adjusting compression applied to the abdomen of the patient by fastening the at least one secondary strap of the elastic compression device over the insert.

28. The method of claim 25, wherein the insert is positioned between the abdomen of the patient and a primary wrap of the elastic compression device.

29. The method of claim 25, further comprising:
adjusting compression delivered to the abdomen via the insert based on one or more of:
adjustment of a primary band of the elastic compression device,
adjustment of at least one secondary strap of the elastic compression device,
adjustment of insert placement, or
adjustment of a moveable component of the insert.

30. The method of claim 25, further comprising:
Iterative application and removal of the insert multiple times during an endoscopic procedure.

31. The method of claim 25, further comprising:
placing a pressure sensor between the abdomen of the patient and the elastic compression device.

32. The method of claim 25, wherein the outer housing includes a wider portion configured for placement at the abdomen of the patient between iliac crests of hips of the patient, wherein the wider portion includes a first edge and a second edge, and wherein the first edge and the second edge of the wider portion taper to a narrower portion configured for the placement at a lower abdominal region below the iliac crests.

33. The method of claim 25, wherein the outer housing includes a first side including a first material, and a second side including a second material and configured for placement toward the abdomen of the patient, wherein the first material is different than the second material, and wherein the second material has a lower hardness than the first material.

34. The method of claim 25, wherein the at least one property of the inner material includes a density within a range of 0.75-1.5 g/cm³.

35. The method of claim 25, wherein the inner material includes one or more of a viscous material, a gel, or a fluidized material.

36. The method of claim 25, further comprising:
inserting at least one of a fluid or a gas into an interior of the outer housing to generate the inner material as a viscous material or change a property of the insert.

37. The method of claim 25, further comprising:
manipulating one or more moveable components within the outer housing to adjust compression to the abdomen of the patient.

38. The method of claim 25, further comprising:
manipulating the inner material of the insert to form a moldable structure to assist in targeted compression at the abdomen.

39. The method of claim 25, wherein the outer housing includes multiple compartments, each compartment filled with one or more inner materials.

40. The method of claim 25, further comprising:
rupturing a container within the outer housing to cause a reaction that warms the insert.
